(19) United States
(12) Patent Application Publication
(10) Pub. No.: US 2014/0303466 A1
(43) Pub. Date: Oct. 9, 2014

(54) Computer-Based Monitoring of Blood Glucose Levels

(71) Applicant: Applivate, LLC, New Haven, CT (US)

(72) Inventors: John Stephen Fitzpatrick, New Haven, CT (US); Clifton Isamu Hayashida, Suffield, CT (US); Harvey Zar, Hamden, CT (US); James McFarland, Newtown, CT (US); Michael Pantalon, Hamden, CT (US); Marianne Sharsky Pantalon, Hamden, CT (US)

(73) Assignee: Applivate, LLC, New Haven, CT (US)

(21) Appl. No.: 14/245,388

(22) Filed: Apr. 4, 2014

Related U.S. Application Data
(60) Provisional application No. 61/809,340, filed on Apr. 6, 2013.

(51) Int. Cl. A61B 5/145; A61B 5/00

(52) U.S. Cl. CPC A61B 5/14532; A61B 5/0022
USPC 600/365

(57) ABSTRACT

Automating the processes of receiving, storing and analyzing blood glucose readings obtained from a diabetic using one or more computer-based systems may provide a number of advantages in the management of levels of glucose in the diabetic's blood. Information regarding a blood glucose reading obtained from a user may be transmitted in one or more messages to the user, to an interested party (e.g. family member), or to a third party over a network, and may serve as the basis for a computer-based motivation strategy to encourage the diabetic to comply with a blood glucose management regime. Additionally, such information may be manually or automatically posted to a social network, thereby enabling the diabetic to receive words of encouragement and support from his or her social network connections, and thereby increase his or her motivation to comply with the blood glucose management regime accordingly.

### CROSS-REFERENCE TO RELATED APPLICATIONS

This application claims the benefit of priority to U.S. Patent Application No. 61/809,340, filed Apr. 6, 2013, the contents of which are incorporated by reference herein in their entirety.

### FIELD OF THE INVENTION

The present invention relates to monitoring blood glucose levels using computer-based equipment and networks.

### BACKGROUND OF THE INVENTION

Diabetes is a serious health condition in which a diabetic's body is unable to regulate the level of glucose in his or her blood. Glucose is one of the main metabolic fuels used by cells in a human body, and insulin is a protein and a hormone produced by the pancreas that promotes the uptake of glucose by such cells. When a body's level of insulin is low, or when the body's cells have become resistant to the effects of insulin, the level of glucose in the blood rises, and the body's cells begin to die from starvation.

Some methods for managing the effects of diabetes provide for monitoring a diabetic's blood glucose level by using a blood glucose meter to periodically obtain a blood glucose reading from the diabetic's blood. The blood glucose reading indicates a level of glucose in the diabetic's blood (e.g., the whole blood or the plasma) at a time when the reading was taken, and may be analyzed with regard to a blood glucose management regime, which may contain a set of requirements within which the blood glucose reading should comport in order to reduce the diabetic's risk of injury or death resulting from unhealthy levels of glucose in his or her blood. Typically, the set of requirements may include a schedule by which blood glucose readings should be taken, or an acceptable range within which a blood glucose level should fall. If a diabetic's blood glucose level is not within the acceptable blood glucose range, the diabetic must take corrective action, e.g., to consume food or receive one or more doses of insulin, in order to raise or lower his or her blood glucose level accordingly.

A level of glucose in a diabetic's blood at any one time may result from a combination of factors relating to the addition of glucose to the blood, the transportation of glucose to cells, or the consumption of glucose according to one ore more metabolic processes. Accordingly, an object of a blood glucose management regime is to balance the effects of these factors, and others, in order to maintain the diabetic's blood glucose levels within an acceptable range. Diabetics use blood glucose management regimes to know when it is necessary to raise their blood glucose levels by eating, e.g., to increase their intake of glucose, or lower their blood glucose levels by administering insulin or other medications, e.g., to increase their uptake, utilization and storage of glucose accordingly.

In order for a blood glucose management regime to provide effective guidance to a diabetic for managing his or her blood glucose levels, the regime should be continuously updated with information regarding current blood glucose readings and strictly adhered to by the diabetic. Adhering to a blood glucose management regime often requires a diabetic to engage in a process sometimes known as “logging,” whereby the diabetic must take and record multiple blood glucose readings throughout a given time period, such as a day or a week. The logged blood glucose readings can then be used to adjust insulin or medication dosages and timing in relation to the diabetic's dietary intake. However, the tedious nature of logging may impede some diabetics, e.g., children or teenagers who may not fully appreciate either the serious nature of their conditions or the importance of logging, from consistently updating information regarding their current blood glucose readings or adhering to a blood glucose management regime. A failure by a diabetic to update information regarding his or her readings, or to adhere to his or her blood glucose management regime, may potentially cause the diabetic to experience serious medical complications that may result in his or her death. Further, a diabetic who is experiencing low blood glucose levels may also suffer from impaired judgment which can impact his or her ability to take appropriate medical actions or to summon help.

### BRIEF DESCRIPTION OF THE DRAWINGS

FIG. 1 is a schematic diagram of a computerized system for monitoring blood glucose levels, in accordance with embodiments of the present disclosure.

FIG. 2 is a block diagram of a computerized system for monitoring blood glucose levels, in accordance with embodiments of the present disclosure.

FIG. 3A and FIG. 3B are block diagrams of a computerized system for monitoring blood glucose levels, in accordance with embodiments of the present disclosure.

FIG. 4 is a flow chart of a process for monitoring blood glucose levels, in accordance with embodiments of the present disclosure.

FIG. 5A and FIG. 5B show a flow chart of a process for monitoring blood glucose levels, in accordance with embodiments of the present disclosure.

FIG. 6 is a flow chart of a process for monitoring blood glucose levels, in accordance with embodiments of the present disclosure.

FIG. 7 is a block diagram of a computerized system for monitoring blood glucose levels, in accordance with embodiments of the present disclosure.

FIG. 8 is a flow chart of a process for monitoring blood glucose levels, in accordance with embodiments of the present disclosure.

FIG. 9 is a user interface displayed by one or more systems or methods for monitoring blood glucose levels, in accordance with embodiments of the present disclosure.

### DETAILED DESCRIPTION

As is set forth in greater detail below, the present disclosure is directed to computer-based systems and methods for monitoring blood glucose levels within a diabetic's body. More specifically, the present disclosure is directed to systems and methods that improve the ability of a diabetic to conduct logging by causing information associated with blood glucose readings obtained from the diabetic to be automatically transmitted to a monitoring system. The monitoring system may receive and analyze the information and send one or more messages regarding the blood glucose readings to the diabetic, as well as to any interested parties associated with the diabetic or to any other third parties.

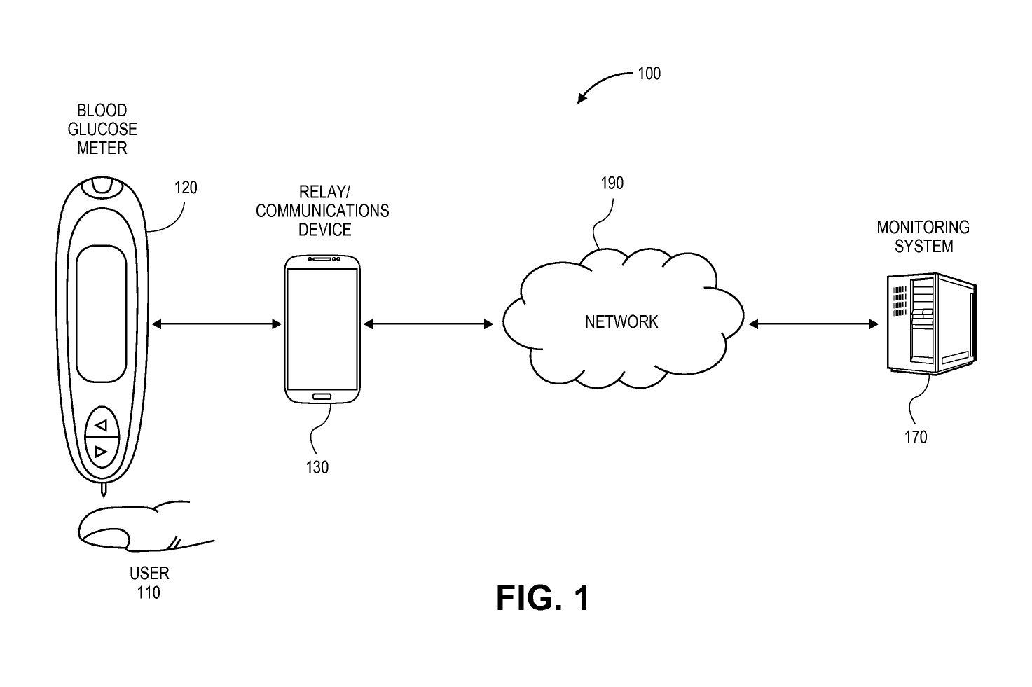

Referring to FIG. 1, components of a computerized system 100 for monitoring blood glucose levels are shown. The system 100 includes a user 110, a blood glucose meter 120 (e.g., a glucometer), a communications device 130 (e.g., a smartphone or other mobile computer device), and a monitoring system 170. The communications device 130 and the monitoring system 170 are configured to communicate with one another over a network 190.

The blood glucose meter 120 may be configured to obtain at least one blood glucose reading from the user 110. As is shown in FIG. 1, the blood glucose meter 120 may obtain a sample of blood from the user 110 through a prick of a body part (e.g., a finger). Alternatively, any other form of blood glucose meter may be utilized in accordance with the present disclosure, including blood-based or bloodless glucose meters. The blood glucose meter 120 or the communications device 130 may be configured to analyze the sample of blood to determine a level of glucose therein or whether the glucose level complies with one or more requirements of a blood glucose management regime. Alternatively, the communications device 130 may be configured to automatically transmit information associated with a blood glucose reading to the monitoring system 170 over the network 190 for analysis.

The monitoring system 170 may be configured to store the information associated with blood glucose readings in a log associated with the user 110, which may be maintained in at least one data store. The monitoring system 170 may be further configured to analyze the information associated with blood glucose readings received from the user 110 over the network 190, and to send messages regarding the at least one blood glucose readings back to the user 110 or to any other parties over the network 190. Such messages may include any relevant information or data regarding the taking of the blood glucose readings, including measured blood glucose levels, times or dates on which such readings were obtained, or any other information regarding the user, as well as words of motivation or encouragement to the user.

Thus, the system 100 for monitoring blood glucose levels shown in FIG. 1 may be used to automate the recording and analysis of information regarding blood glucose readings, or the distribution of such information to one or more individuals or entities.

The effectiveness of a blood glucose management regime that has been defined for a user, or assigned to the user, can be critically improved by regularly providing the blood glucose management regime with information regarding blood glucose readings that have been recently taken from the user. For example, such information may be used to adjust insulin or medication dosages and timing in relation to a diabetic's dietary intake, in order to increase the extent to which a diabetic may control his or her blood glucose levels. In some situations, the failure by a diabetic to provide a blood glucose management regime with up-to-date information regarding his or her blood glucose levels may lead to serious medical complications for the diabetic, and may even result in his or her death. Therefore, adhering to the requirements of a blood glucose regime often requires a diabetic to actively and diligently measure and record his or her blood glucose readings throughout a given time period.

Previously, the process of logging was accomplished by manually recording blood glucose readings into a paper log, or manually entering such readings into one or more computerized systems. For example, some computerized logging systems require a user to manually type and send an alphanumeric message to an external server, or otherwise provide a computer application with a manual input after the blood glucose readings have been obtained, thereby enabling such information regarding the blood glucose readings to be transmitted to a server. The additional steps that are required to identify information regarding a blood glucose reading, and to manually record such information into a paper log or manually enter such information into one or more computers, contribute to the tedious nature of logging, which may impede some diabetics from fully participating in a blood glucose management regime, or adhering to one or more requirements of such a regime. Of particular concern are children and/or teenaged diabetics who may not yet recognize the dangers associated with failing to properly monitor or manage their conditions, or fully understand the importance of logging, or elderly persons who may be unable or unwilling to perform the various tasks in order to properly log information regarding their conditions.

While automating the recording and analysis of blood glucose readings might simplify the task of logging, a diabetic must take such blood glucose readings on a regular basis, and demonstrate the commitment and will to adhere to a blood glucose management regime, in order to safely minimize the probability that he or she will develop medical complications due to his or her diabetic condition. Moreover, while a diabetic who is motivated to adhere to a blood glucose management regime is more likely to do so than an unmotivated diabetic, maintaining such motivation may be challenging for some diabetics, particularly children or teenagers diabetics who, as is discussed above, may not yet fully understand the dangers associated with mismanagement of their conditions or the critical importance of logging to the success of a blood glucose management regime.

Accordingly, the systems and methods disclosed herein may automate the processes of obtaining, recording and analyzing information regarding one or more blood glucose readings, and may transmit one or more messages comprising such information to the user, or to one or more interested parties (e.g., parents or other family members) or to other third parties (e.g., physicians, medical staff or other diabetics). Some embodiments of the present disclosure may further incentivize a diabetic to actively participate in logging, or to strictly comply with a blood glucose management regime, by causing one or more awards to be distributed to a diabetic for having timely taken his or her blood glucose readings, or for having adhered to the requirements of a blood glucose management regime. Such awards may be selected in accordance with a motivation strategy. Because the effectiveness of a particular award or a motivation strategy to incentivize a particular diabetic may change over time, the systems and methods of the present disclosure may also detect a change, e.g., a decrease, in the effectiveness of the particular award and/or motivation strategy over time, and alter a type of award or a motivation strategy used to incentivize the diabetic. For example, when a monitoring system detects that a teenaged diabetic has taken five blood glucose readings, and that each of the readings is compliant with one or more requirements of a blood glucose management regime, the monitoring system will notify the diabetic that he or she is eligible to receive a one-dollar ($1) credit toward a purchase of new music from an online store.

Conversely, some embodiments of the present disclosure may detect a diabetic's failure to adhere to a blood glucose management regime, and if necessary, transmit one or more messages to not only the diabetic but also any interested parties who may be associated with the diabetic (e.g., parents or other family members of the diabetic), or any other third parties (e.g., physicians, medical personnel or other diabetics), indicating that the diabetic has failed to comply with one or more requirements of the blood glucose management regime. With the knowledge that interested parties and/or other parties will be notified when a diabetic fails to adhere to a blood glucose management regime, the diabetic may be further motivated to timely take his or her blood glucose readings and to comply with the requirements of the blood glucose management regime. For example, when a ten-year-old diabetic fails to take a prescribed medication as scheduled, and subsequently takes a blood glucose reading using a blood glucose meter, information associated with the blood glucose reading may be automatically transmitted from a communications device associated with the blood glucose meter to a monitoring system. Upon receiving the information, the monitoring system analyzes the information, detects that the ten-year-old's blood glucose level is above a target range specified in a relevant blood glucose management regime, and sends a warning message to the diabetic, to her parents, and to her doctor. After receiving the warning message, the parents of the ten-year-old may call her by telephone or speak with her directly to express their disappointment or to remind her of the importance of taking her medication.

Some other embodiments of the present disclosure may transmit one or more electronic messages or other notifications to interested parties or to third parties indicating that a diabetic's failure to adhere to a blood glucose management regime poses a significant health risk to the diabetic, or that the diabetic requires medical assistance. For example, when a seventy-five-year-old diabetic fails to take his medication as scheduled on a Monday morning, the diabetic may enter diabetic shock and collapse. Subsequently, a monitoring system may detect that it has not received a blood glucose reading from the diabetic since Sunday night and transmit warning messages to the diabetic, to his children, to his doctor or to an emergency medical service. Upon receiving such messages, the diabetic's children or medical personnel may rush to his side and administer his medication, thereby saving his life.

The system and methods of the present disclosure may also improve the ability of a diabetic to control his or her blood glucose level by making predictions as to the diabetic's blood glucose level based on one or more attributes, and providing one or more messages to the diabetic, to interested parties associated with the diabetic or to other parties regarding such predictions. For example, a monitoring system might receive information concerning a blood glucose reading obtained from a diabetic indicating that his blood glucose level spiked after dining at a clam shack around lunchtime on Monday. The monitoring system then receives additional information around lunchtime on Friday that the diabetic is in a vicinity of the clam shack. Based upon the previously received information regarding the spike in the diabetic's blood glucose level on Monday, the monitoring system causes a message to be sent to the diabetic, or to his wife and to his doctor, indicating that the diabetic's blood glucose level spiked the last time he was near the clam shack around lunchtime. After being made aware of this information, the diabetic may be prompted to take more insulin when dining at the clam shack, or when eating seafood in general.

The system and methods disclosed herein may further improve the ability of a diabetic to adhere to a blood glucose management regime by allowing the diabetic to draw motivational support from interested parties associated with the diabetic and/or other diabetics. For example, where a teenaged diabetic has been struggling to adhere to a blood glucose management regime, the teenaged diabetic may post one or more messages to a social media outlet expressing frustration with her inability to adhere to the blood glucose management regime. The diabetic may then receive messages of encouragement from her friends, from members of her family or from other diabetics who may share similar characteristics with the teenaged diabetic (e.g., diabetics of the same gender, or of the same or similar ages or locations). Similarly, when the diabetic obtains a blood glucose reading that is compliant with the requirements of the blood glucose management regime, the systems and methods disclosed herein may make a post to the social media outlet on behalf of the diabetic reporting her compliant blood glucose reading, and her friends, family members or other diabetics may send her congratulatory messages by way of the social media outlet. Motivated by the praise she receives from her friends, from her family or from other diabetics over social media, the diabetic may be willing to continue to adhere to the requirements of the blood glucose management regime.

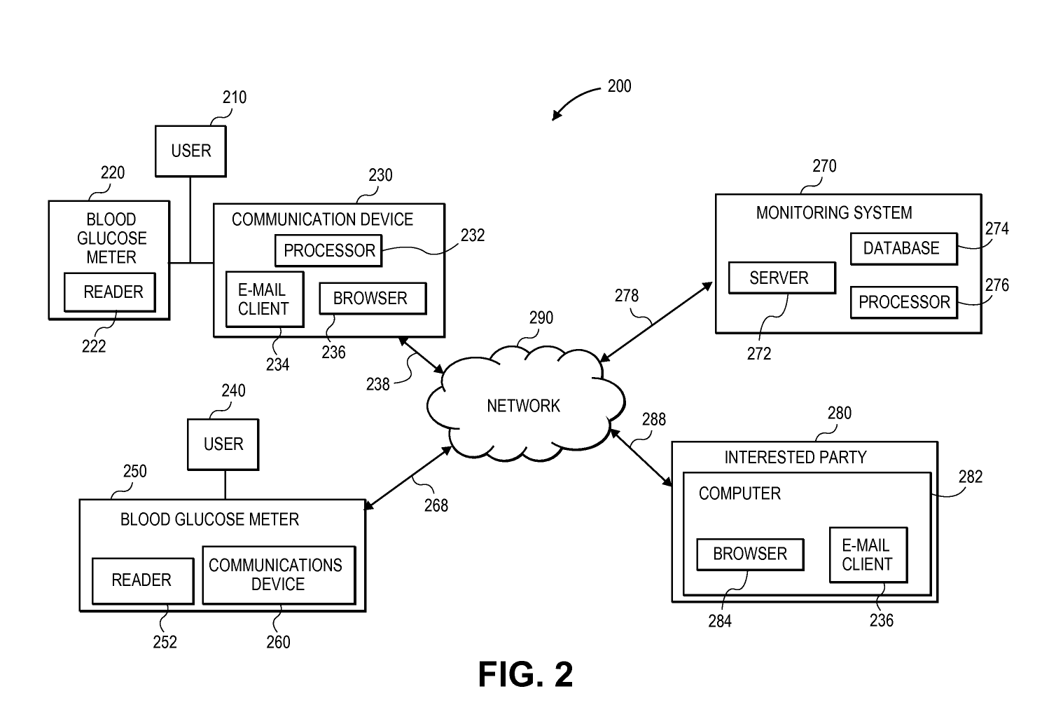

Referring to FIG. 2, a block diagram of a computerized system 200 for monitoring blood glucose levels is shown. The system 200 includes users 210, 240, blood glucose meters 220, 250, communications devices 230, 260, a monitoring system 270, and an interested party 280. The communications devices 230, 260, the monitoring server 270, and the interested party 280 are configured to communicate with each other over a network 290.

The users 210, 240 may have a type of diabetes or another diabetic condition that requires the monitoring of levels of glucose and/or insulin in their blood. For example, the users 210, 240 may have one or more metabolic diseases or conditions characterized by high levels of glucose in the blood, including but not limited to Type 1 diabetes, Type 2 diabetes or gestational diabetes.

The blood glucose meters 220, 250 include readers 222, 252 that may be configured to measure the level of glucose in a blood sample captured from the users 210, 240. For example, the readers 222, 252 may be configured to determine a concentration of glucose within a sample taken from one of the users 210, 240, e.g., by a prick of a finger, such as is shown in FIG. 1, and placed onto a glucose meter test strip or like materials for subsequent evaluation. Alternatively, the readers 222, 252 may operate in a bloodless manner, and may determine or estimate a blood glucose level by spectroscopic or other non-invasive means. The blood glucose meters 220, 250 and/or the communications devices 230, 260 may be configured to determine levels or concentrations of glucose in samples obtained by the readers 222, 252, and to communicate information regarding such levels or concentrations with the communications devices 230, 260. Additionally, the blood glucose meters 220, 250 and/or the communications devices 230, 260 may also be further configured to determine whether such levels or concentrations comply with one or more requirements of a blood glucose management regime.

The communications devices 230, 260 may include one or more processors 232 and at least one means for communicating over the network, including an email client 234 and a browser 236. The communications devices 230, 260 may be configured to connect to the network 290, as indicated by lines 238, 268, in order to transmit and receive information and messages associated with blood glucose readings to and from the monitoring system 270, in the form of digital or analog data, or for any other purpose. Additionally, as is shown in FIG. 2, the blood glucose meters and the communications devices of the present disclosure may be provided as discrete units in communication with one another, such as the blood glucose meter 220 and the communications device 230 shown in FIG. 2, such that the communications devices may receive information regarding blood glucose readings taken using the blood glucose meters and transmit such information to one or more computer devices or entities over the network.

In this regard, the communications devices 230, 260 may be any form of computer-based device for communicating over the network 290, including but not limited to a general purpose computer such as a smartphone, a tablet computer, a laptop computer or a desktop computer, or a computer specifically adapted for one or more of the purposes described herein. Alternatively, as is also shown in FIG. 2, the blood glucose meters and the communications devices of the present disclosure may be integrated into a single unit, such as the blood glucose meter 250 having the reader 252 and the communications device 260. For example, the blood glucose meter 250 may be equipped with one or more wireless or wired communications devices (e.g., cellular or other wireless communication transceivers) to communicate directly with the network 290.

Alternatively, the communications devices 230, 260 may be configured to use an intermediate relay device (not shown) to connect to the network 290. For example, the communications devices 230, 260 may receive information regarding one or more blood glucose readings from the blood glucose meters 220, 250, and transmit such information to a relay device in the form of a general purpose computer such as a smartphone, a tablet computer, a laptop computer or a desktop computer, or a computer specifically adapted for one or more of the purposes described herein, which may then transmit such information to another computer device over the network 290.

As is shown in FIG. 2, the monitoring system 270 includes a server 272, a database 274 and at least one processor 276. The monitoring system 270 may be configured to connect to the network 290, as indicated by line 278, and to send and receive information or messages associated with blood glucose readings obtained from the users 210, 240 in the form of digital or analog data, or for any other purposes. The database 274 may be configured to store any information or data regarding the users 210, 240, as well as blood glucose readings taken from the users 210, 240, or any information or data of any type or form. The database 274 may be provided in conjunction with the same physical and/or virtual machine as the server 272 or, alternatively, on a different physical and/or virtual machine, in accordance with the present disclosure.

As is also shown in FIG. 2, the interested party 280 may be one or more individuals or entities having an express or implied interest in a blood glucose level of the users 210, 240, such as a parent or other family member of the users 210, 240. The interested party 280 may operate one or more computers 282, which may have one or more software applications associated therewith, including a browser 284 and/or an electronic mail (or “E-mail”) client 236. The interested party 280 and/or the computer 282 may be configured to connect to the network 290, as indicated by line 288, in order to transmit and receive information and messages associated with blood glucose readings obtained from the users 210, 240, to and from the monitoring system 270, in the form of digital or analog data, or for any other purpose. Additionally, the system 200 of FIG. 2 may further include one or more other third parties, such as physicians, medical staff or other diabetics (not shown), who may also be interested in communicating with the users 210, 240 over the network 290 by way of one or more computer devices or applications available thereon, in a manner similar to that of the interested party 280.

The blood glucose meters 220, 250 and the communications devices 230, 260 or relay devices may be configured to communicate with each other by way of one or more physical or wireless connection paths. For example, such connection paths may include one or more Universal Serial Bus (or “USB”) cables, serial cables, shared buses, and/or any other type of physical connection paths. Such connection paths may also include personal area networks (“PANs”), including such PANs operating in accordance with any form of communications protocol, including but not limited to Bluetooth or Wireless Fidelity (or “WiFi”), as well as direct wireless links (e.g., infrared, laser, radio or the like) and/or other types of wireless connection paths.

The computers, servers, devices and the like described herein have the necessary electronics, software, memory, storage, databases, firmware, logic/state machines, microprocessors, communication links, displays or other visual or audio user interfaces, printing devices, and any other input/output interfaces to provide any of the functions or services described herein and/or achieve the results described herein. Also, those of ordinary skill in the pertinent art will recognize that users of such computers, servers, devices and the like may operate a keyboard, keypad, mouse, stylus, touch screen, or other device (not shown) or method to interact with the computers, servers, devices and the like, or to “select” an item, link, node, hub or any other aspect of the present disclosure.

Those of ordinary skill in the pertinent arts will understand that process steps described herein as being performed by a “blood glucose meter,” a “reader,” a “communications device,” a “relay device,” a “monitoring system,” a “user” (or a “diabetic”), an “interested party,” a “third party” or like terms may be automated steps performed by their respective computer systems, or implemented within software modules (or computer programs) executed by one or more general purpose computers. Moreover, process steps described as being performed by a “blood glucose meter,” a “reader,” a “communications device,” a “relay device,” a “monitoring system,” a “user” (or a “diabetic”), an “interested party,” a “third party” or like terms may be typically performed by a human operator, but could, alternatively, be performed by an automated agent.

The blood glucose meters 220, 250, the communications devices 230, 260, the relay devices, the monitoring system 270, the users 210, 240, and/or the interested party 280 or third parties disclosed herein may use any web-enabled applications or features, or any other client-server applications or features including E-mail, or other messaging techniques, to connect to the network 290 or to communicate with one another, such as through short or multimedia messaging service (SMS or MMS) text messages. For example, the monitoring system 270 may be adapted to transmit information or data in the form of synchronous or asynchronous messages to the interested party 280 and/or the communications devices 230, 260 or any other computer device in real time or in near-real time, or in one or more offline processes, via the network 290. Those of ordinary skill in the pertinent art would recognize that the monitoring system 270, the interested party 280, the third parties or the users 210, 240 may operate any of a number of computing devices that are capable of communicating over the network, including but not limited to set-top boxes, personal digital assistants, digital media players, web pads, smartphones, tablet computers, laptop computers, desktop computers, electronic book readers, and the like. The protocols and components for providing communication between such devices and over the network 290 are well known to those skilled in the art of computer communications and need not be described in detail herein.

The data and/or computer executable instructions, programs, firmware, software and the like (also referred to herein as “computer executable” components) described herein may be stored on a computer-readable medium that is within or accessible by computers or computer components such as the blood glucose meters 220, 250, the communications devices 230, 260, the monitoring system 270, or any other computers or control systems utilized by the monitoring system 270, the users 210, 240, the third parties or the interested party 280 and having sequences of instructions which, when executed by a processor (e.g., a central processing unit, or “CPU”), cause the processor to perform all or a portion of the functions, services and/or methods described herein. Such computer executable instructions, programs, software and the like may be loaded into the memory of one or more computers using a drive mechanism associated with the computer readable medium, such as a floppy drive, CD-ROM drive, DVD-ROM drive, network interface, or the like, or via external connections.

Some embodiments of the systems and methods of the present disclosure may also be provided as a computer executable program product including a non-transitory machine-readable storage medium having stored thereon instructions (in compressed or uncompressed form) that may be used to program a computer (or other electronic device) to perform processes or methods described herein. The machine-readable storage medium may include, but is not limited to, hard drives, floppy diskettes, optical disks, CD-ROMs, DVDs, ROMs, RAMs, erasable programmable ROMs (“EPROM”), electrically erasable programmable ROMs (“EEPROM”), flash memory, magnetic or optical cards, solid-state memory devices, or other types of media/machine-readable medium that may be suitable for storing electronic instructions. Further, embodiments may also be provided as a computer executable program product that includes a transitory machine-readable signal (in compressed or uncompressed form). Examples of machine-readable signals, whether modulated using a carrier or not, may include, but are not limited to, signals that a computer system or machine hosting or running a computer program can be configured to access, or including signals that may be downloaded through the Internet or other networks.

For example, the communications devices 230, 260 may be in communication with the blood glucose meters 220, 250 over a USB cable, while the communications device or relay device may comprise a cellular-enabled device such as a smartphone or tablet. Additionally, the communications devices 230, 260 may be configured to communicate with one or more relay devices over a Bluetooth connection, a WiFi connection, or over any wired or wireless connection. The relay devices may operate a relay application that facilitates the automatic transmission of the information and messages regarding the blood glucose readings obtained from the users 210, 240 between the blood glucose meters 220, 250 and the monitoring system 270. Such a relay application may be configured to start up automatically upon a powering up of the relay device, or, alternatively, may be initiated by one of the users 210, 240 prior to obtaining a blood glucose reading. When a user 210, 240 is required to open the relay application prior to obtaining a blood glucose reading, a relay application may be configured to remain open after the blood glucose reading has been transmitted, or further configured to facilitate the automatic transmission or receipt of information and messages regarding subsequent blood glucose readings obtained by the users 210, 240, between the blood glucose meters 220, 250 and the monitoring system 270 for as long as the relay application remains open. Additionally, a relay application may be further configured to remain open until the users 210, 240 closes it and/or the relay device powers down. A similar application may reside on the communications devices 230, 260.

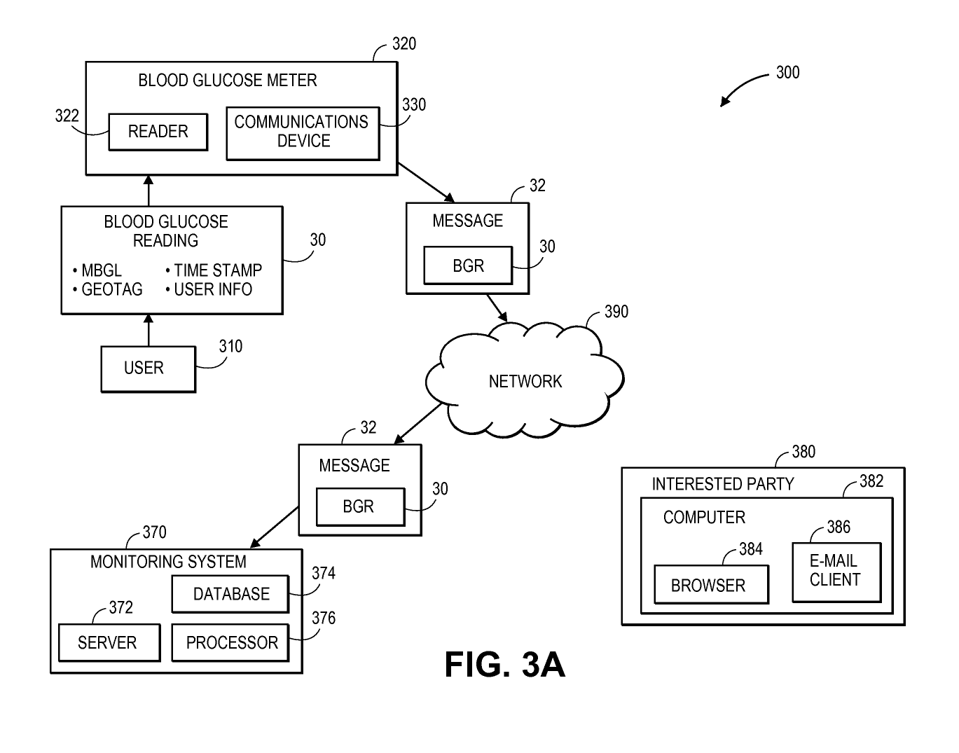

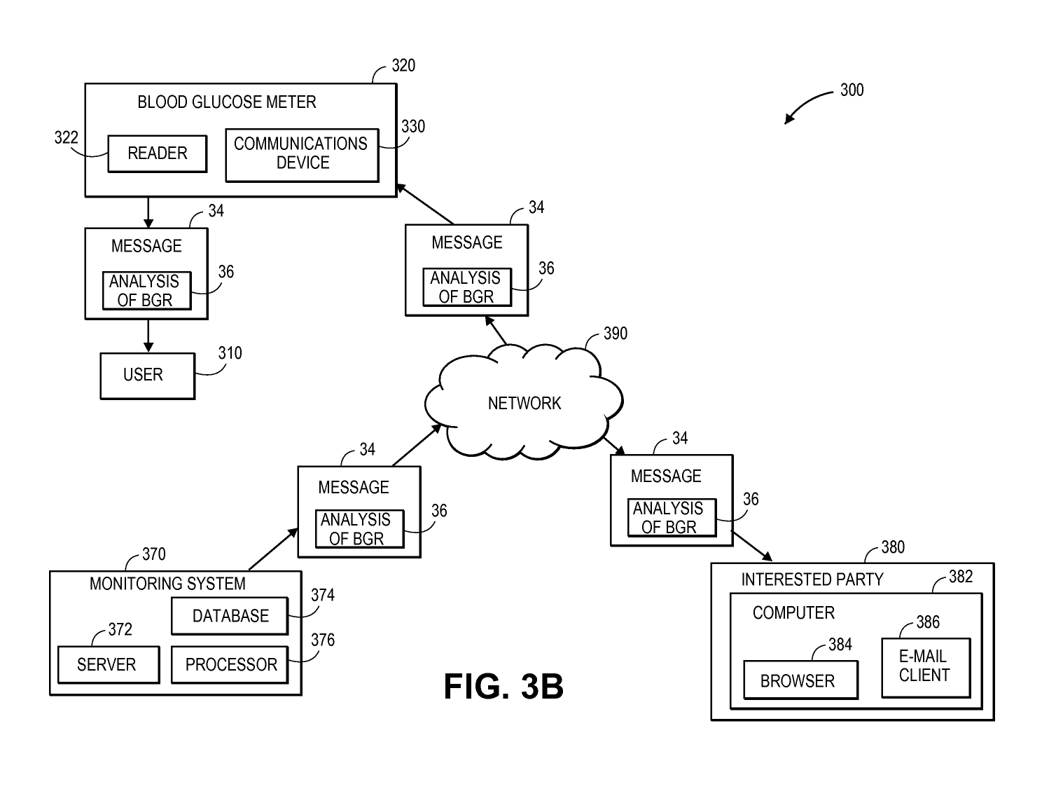

As is discussed above, the systems and methods of the present disclosure may be provided to capture blood glucose readings from a user, to transmit information regarding one or more of such readings to a monitoring system, to process and store such information, and to transmit at least some of the information in one or more messages to the user, to one or more interested parties (e.g., parents or other family members of the diabetic), or to any other third parties (e.g., physicians, medical personnel or other diabetics). Referring to FIG. 3A and FIG. 3B, block diagrams of a computerized system 300 for monitoring blood glucose levels are shown. Referring to FIG. 3A, a blood glucose reading 30 may be obtained from a user 310 by using a blood glucose meter 320 having a reader 322. The blood glucose reading 30 may include a blood glucose level of the user, as measured by the reader 322 (e.g., a “MBGL”), as well as other information or data regarding the blood glucose reading 30, including but not limited to a timestamp indicating a time or a date on which the blood glucose reading 30 was obtained from the user 310, a geotag indicative of a location at which the blood glucose reading 30 was obtained from the user 310 (e.g., coordinates, network access point locations, addresses, geographic identifiers or any other information referencing a geographic location, which may be determined by a Global Positioning System or other computer-based system, as well as a manual entry), or any other intrinsic or extrinsic user-generated or user-related information or data associated with the blood glucose reading 30 or the user 310.

For example, the user information may identify an emotional state of the user 310 at a time when the blood glucose reading 30 was obtained, and may thus indicate whether the user 310 was proud, angry, sad, happy, stressed, frustrated, motivated, satisfied or in another emotional state at that time. The user information may also identify a physical state of the user 310 at a time when the blood glucose reading 30 was obtained, and may thus indicate whether the user 310 was hungry, full, tired, energetic, awake, asleep, healthy, sick, hot, cold, or in another physical state at that time. The user information may further identify a categorical type of food associated with the blood glucose reading 30. For example, the user information may indicate that the blood glucose reading 30 was consistent with the consumption of food in one or more categories, e.g., Chinese food, Italian food, French food, German food, fast food, fruits, vegetables, grains, fats or food of any other category.

Upon obtaining a blood glucose reading 30 from the user 310 using the blood glucose meter 320, the communications device 330 automatically transmits a message 32 including information associated with the blood glucose reading 30 over a network 390 to a monitoring system 370. Additionally, where the blood glucose reading 30 has been compared to one or more requirements of a blood glucose management regime at the blood glucose meter 320, the message 32 may further include information indicating whether the blood glucose reading 30 is compliant, or whether further readings are required. The monitoring system 370 may include one or more processors 376 and operate one or more monitoring applications that may be configured to receive the message 32 and the information associated with the blood glucose reading 30 at the server 372, and to store the received information 32 in at least one database 374. The processors 376 may further adapt the server 372 to conduct one or more analyses of the information associated with the blood glucose reading 30 set forth in the message 32.

Referring to FIG. 3B, upon completing an analysis of the information regarding the blood glucose reading 30, the monitoring system 370 may generate and transmit one or more electronic messages 34 including information regarding the analysis 36 of the blood glucose reading 30 to the user 310, to an interested party 380 or to any other parties over the network 390. The messages 34 may be transmitted and received via E-mail, text messages or any other known form of electronic communication, and in accordance with one or more communication protocols.

Accordingly, the systems and methods of the present disclosure may automate processes ordinarily associated with logging, including the taking one or more blood glucose readings from a user, the transmission of information regarding such readings (e.g., measured blood glucose readings, timestamps, geotags or any other user-related information) to a monitoring system, recording or otherwise storing such information in at least one data store, and transmitting electronic messages regarding the blood glucose readings to the user, to an interested party associated with the user, or to one or more third parties.

Additionally, as is discussed above, the systems and methods of the present disclosure may further determine whether a diabetic has adhered to a blood glucose management regime, and, when necessary, transmit one or more messages to the diabetic, to interested parties associated with the diabetic or to other third parties regarding the extent to which the diabetic has complied with the blood glucose management regime. Moreover, where a diabetic's failure to adhere to a blood glucose management regime may pose a significant health risk to the diabetic, messages may be transmitted to the diabetic, or to such interested parties or third parties, indicating that the diabetic requires medical assistance.

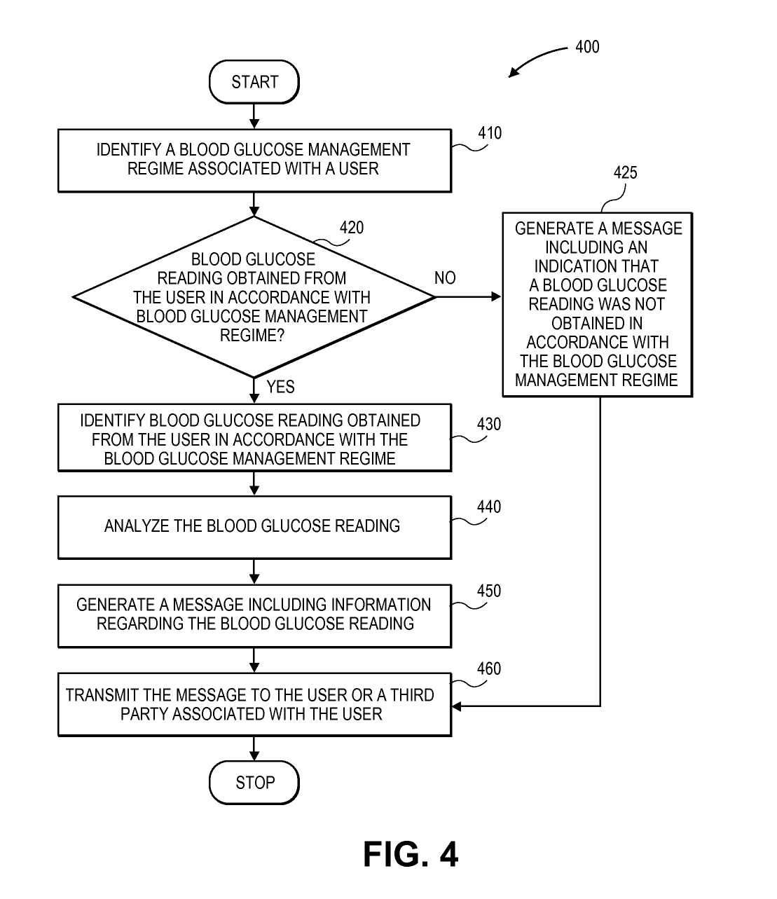

Referring to FIG. 4, a flow chart 400 of one process for monitoring blood glucose levels is shown. At box 410, a blood glucose management regime associated with a user is identified. The blood glucose regime may include one or more requirements, including a mandatory schedule according to which blood glucose readings must be obtained from the user, as well as preferred ranges within which the user's measured blood glucose levels should preferably remain. The blood glucose management regime may be specifically tailored to one or more particular attributes of the user, such as the severity of his or her diabetic condition, as well as his or her history in complying with blood glucose management regimes or other treatment programs. Alternatively, the blood glucose management regime may be generally prescribed for the user based on the user's age, gender, weight or height, or other characteristics. Information regarding the blood glucose management regime may be stored in one or more databases associated with the monitoring system, or supplied to the monitoring system from an external data source. Additionally, the blood glucose management regime may be a previously established regime, or it may be assembled in real time or in near-real time based on one or more attributes of the user. The blood glucose management regime may be associated with the user by a database key, which may be an identification number, a user name or any other code or set of alphanumeric symbols that may be used to associate database records with users.

At box 420, whether a blood glucose reading has been obtained from the user in accordance with the blood glucose management regime is determined. For example, a blood glucose management regime may include a requirement that a blood glucose reading obtained from a user must be received by 9 o'clock in the morning on a Monday. If the monitoring system determines that a blood glucose reading was obtained from the user by 9 o'clock in the morning on Monday, or within a predetermined range of time before or after 9 o'clock in the morning, then the user has satisfied the requirement of the blood glucose management regime. If the monitoring system determines that a blood glucose reading was never obtained from the user, or that the blood glucose reading was obtained outside of the predetermined range of time (e.g., too early or too late), then the user has not satisfied the requirement of the blood glucose management regime.

If a blood glucose reading obtained from the user has not been received in accordance with the blood glucose management regime, then the process advances to box 425, where a message indicating that a blood glucose reading was not obtained in accordance with the blood glucose management regime is generated, and to box 460, where the message is transmitted to the user, or to interested parties associated with the diabetic (e.g., parents or other family members), or any other third parties (e.g., physicians, medical personnel or other diabetics). For example, upon determining that a blood glucose reading was not obtained from a user, or that the blood glucose reading was not obtained within a predetermined range of time, a monitoring system may generate and transmit messages instructing the user to have his or her blood glucose reading taken, or informing the user's family members or primary care physician that the user has not complied with one or more requirements of the blood glucose management regime. Such messages may be transmitted by any known electronic messaging means, including E-mail, SMS or MMS text messages, or the like.

If a blood glucose reading was obtained from the user in accordance with the blood glucose management regime, the process advances to box 430, where the blood glucose reading obtained in accordance with the blood glucose management regime is identified, and to box 440, where the blood glucose reading is analyzed. The blood glucose reading may have been received from a blood glucose meter, such as the blood glucose meter 120 of FIG. 1, and transmitted to the monitoring system 170 over the network 190, and stored in any type or form of data store, such as a database, a hard drive, a removable memory card, or the like, and associated with the user.

At box 440, the monitoring system analyzes the received blood glucose reading. The analysis may identify one or more elements of the blood glucose reading, and compare such elements to one or more requirements of the blood glucose management regime. If the elements of the blood glucose reading satisfy the requirements of the blood glucose management regime, the blood glucose reading may be considered to be in accordance with the blood glucose management regime. If one or more of the elements of the blood glucose reading do not satisfy the requirements of the blood glucose management regime, then the blood glucose reading may be considered to not be in accordance with the blood glucose management regime.

For example, a blood glucose management regime may include a requirement that a measured blood glucose level of a user must remain between 80 mg/dl and 120 mg/dl. When a blood glucose reading obtained from the user is received by the monitoring system, the monitoring system may identify a measured level of glucose in the user's blood, and compare the measured blood glucose level to the range specified in the requirement. If the measured blood glucose level is within the specified range of 80 mg/dl to 120 mg/dl, then the blood glucose reading may be determined to comply with the blood glucose management regime. If the measured blood glucose level is less than 80 mg/dl, or greater than 120 mg/dl, then the blood glucose reading does not comply with the blood glucose management regime.

At box 450, a message including information regarding the blood glucose reading is generated. For example, if a blood glucose reading obtained from a user and received by a monitoring system does not comply with one or more requirements of a blood glucose management regime, e.g., if a measured blood glucose level of the user falls outside a specified range, then the message may include an indication that the blood glucose reading does not comply with the blood glucose management regime, and that the user must consume food, or take insulin, as necessary, in order to address the non-compliant blood glucose level. If the blood glucose reading obtained from the user and received by the monitoring system complies with all relevant requirements of the blood glucose management regime, e.g., if the measured blood glucose level of the user is within the specified range, then the message may include an indication that the blood glucose reading complies with the blood glucose management regime.

At box 460, the message is transmitted to the user or an interested party associated with the user, e.g., by E-mail, SMS or MMS text message, or any other form of electronic communication. For example, a text message regarding the analysis of the blood glucose reading may be sent to the user, while an E-mail message summarizing the analysis of the blood glucose reading may be delivered to an account associated with the user's parents or family members, or to a support group of which the user is a member, and an internal message may be transmitted to a record-keeping system maintained by the user's physician. Upon receiving the message, the user may elect to address his or her blood glucose levels in any appropriate manner (e.g., by taking insulin or consuming food), while interested parties or third parties may take note of the user's blood glucose levels and respond in kind, such as with a telephone call to the user, or by summoning medical assistance, as necessary.

Accordingly, the systems and methods of the present disclosure may determine whether a user has complied with one or more requirements of a blood glucose management regime, including by confirming that the user has timely obtained one or more blood glucose readings, or that the blood glucose levels within such readings are satisfactory, and transmitting one or more messages to the user, or to interested parties or third parties, as necessary, including information regarding such blood glucose readings.

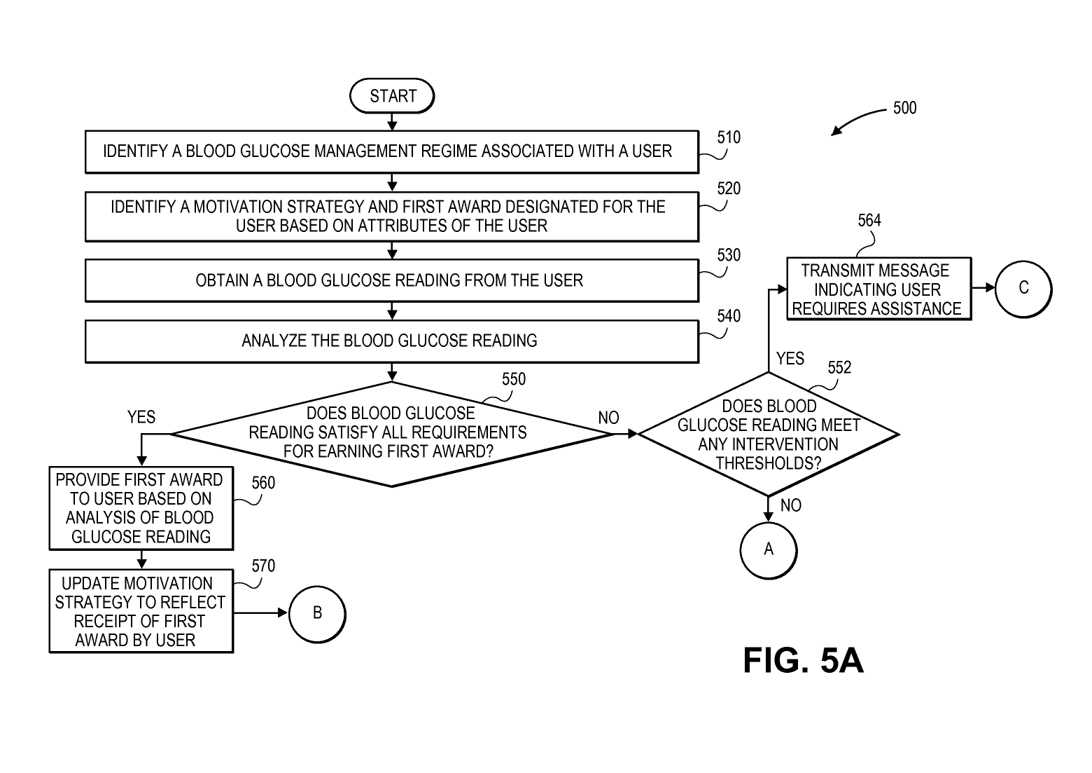

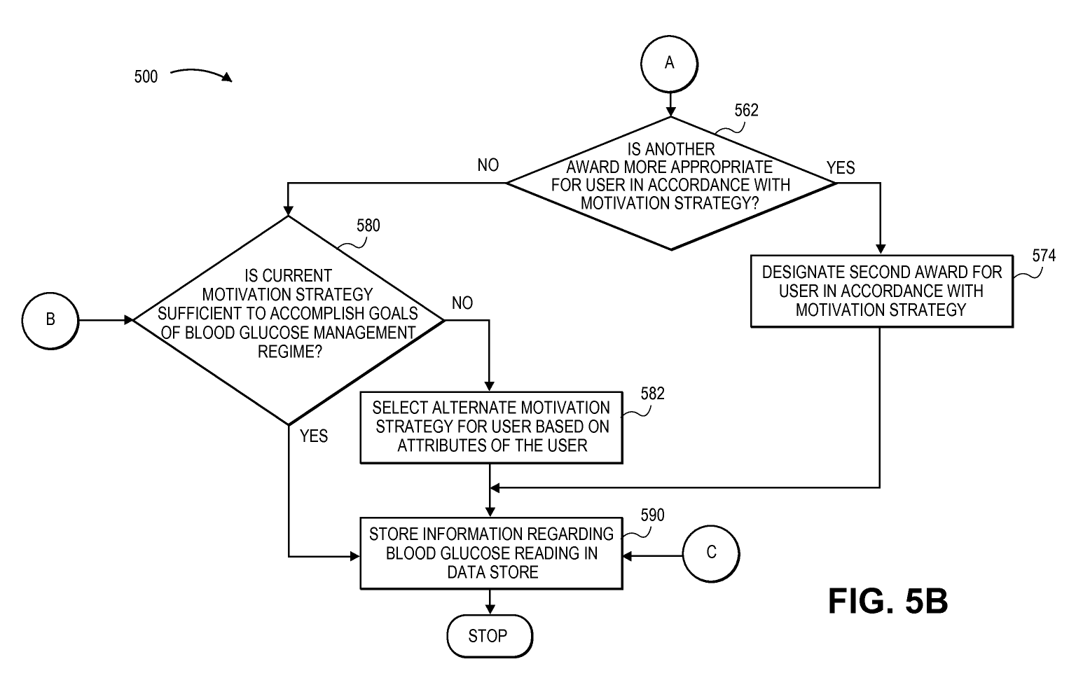

As is also discussed above, the systems and methods of the present disclosure may define a motivation strategy that provides one or more awards or incentives to a diabetic, in order to encourage the diabetic to comply with requirements of a blood glucose management regime. The motivation strategy may be customized for a user, based on factors such as the severity of his or her diabetic condition, or his or her history in complying with blood glucose management regimes or other treatment programs, or may be generally prescribed for the user based on his or her similarity to one or more other users who have previously participated in a blood glucose management regime. Referring to FIG. 5A and FIG. 5B, a flow chart of a process 500 for monitoring blood glucose levels is shown. At box 510, a blood glucose management regime associated with a user is identified. The blood glucose management regime may have been assigned to the user by a physician or an interested party (e.g., a parent or other family member), or voluntarily entered into by the user. Information regarding the blood glucose management regime of the user may be identified based on a UserID, an Internet Protocol (or “IP”) address, a telephone number, and/or any other alphanumeric string that may be associated with the user.

At box 520, a motivation strategy is identified and designated for the user based on one or more attributes of the user. Such attributes may include, but are not limited to, an age, a race, a gender, a hobby, a height, a weight or a location of the user, as well as a specific diabetic condition with which the user has been diagnosed, any family history or genetic predisposition of the user to the diabetic condition, a medication prescribed to the user, or any medical devices or equipment that may be available to the user. Additionally, the motivation strategy may include one or more predefined goals (e.g., maintaining a blood glucose level within a predetermined range, or above or below a given threshold), as well as one or more awards or incentives, of various types, that may be awarded to the user. Such awards may be provided for the purpose of motivating the user to adhere to the blood glucose management regime, and to ultimately attain the goals.

For example, where the user is a child, and a goal defined for the user is to have the user obtain blood glucose readings in accordance with a schedule, the motivation strategy may include non-monetary awards that may be configured to make the process of obtaining blood glucose readings fun by providing instant gratification to the user when he or she obtains a blood glucose reading. Accordingly, a motivation strategy may include awards or incentives such as badges, videos or age-appropriate educational information. If the user is a teenager, a motivation strategy may include awards configured to provide positive reinforcement during the adherence to a blood glucose management regime, and to encourage the user to work toward long-term goals. Some awards or incentives provided in accordance with such a regime may include an increase in an allowance, credit or gift cards for purchasing online media or similar gifts, as well as words of encouragement or praise for reaching a goal by E-mail or text message, or customized awards defined by the user or an interested party. Similarly, if the user is an elderly adult, the motivation strategy may distribute awards not only to the user but also to an interested party, in order to encourage the interested party to confirm that a blood glucose reading was obtained from the elderly user.

At box 530, a blood glucose reading is obtained from the user, and at box 540, the blood glucose reading is analyzed. For example, one or more elements in the blood glucose reading may be obtained and compared to requirements included in the blood glucose management regime, such as a measured level of glucose in plasma or whole blood, a geotag consistent with a location where the blood glucose reading was obtained, a time stamp indicative of a time or date when the blood glucose reading was obtained, and user-specific or user-generated information, as well as any other relevant information. At box 550, whether the blood glucose reading satisfies all of the requirements for earning a first award in accordance with the motivation strategy is determined.

If the blood glucose reading satisfies all of the requirements for earning the first award, then the process advances to box 560, where the first award is provided to the user based on the analysis of the blood glucose reading, and to box 570, where the motivation strategy is updated to reflect the receipt of the first award by the user. For example, where a monitoring system determines that a user has timely obtained each of his or her blood glucose readings over a predefined period of time (e.g., a week, a month or a year) or number of readings (e.g., ten, twenty or thirty consecutive readings), as defined by a motivation strategy, the monitoring system may then determine that the user has earned the first award. Similarly, where each of the user's blood glucose levels remains within a predetermined range (e.g., between 80 and 120 mg/dl) over a predefined period of time or number of readings, as defined by a motivation strategy, the monitoring system may then determine that the user has earned the first award. Conversely, if the user has not timely obtained his or her blood glucose readings, or if the blood glucose levels identified in such readings do not fall within a predetermined range, the monitoring system may determine that the user has not earned a first award.

Awards or incentives may be provided to users by any means in accordance with the present disclosure. For example, referring to FIG. 5A and FIG. 5B, the first award may be provided to the user by way of an electronic message that contains hyperlinks or other information that permit the user to access the first award, e.g., a gift card or discount available for use at one or more electronic commerce websites. Alternatively, the first award may be directly provided to the user, e.g., by mail or by direct deposit. Moreover, non-monetary awards or incentives may also be provided, such as the right of a teenager to use a parent's car, and one or more electronic messages may be transmitted to a user, or to interested parties or third parties, when the monitoring system determines that the user has earned the first award. Eventually, subsequent awards may be distributed with less frequency as the user becomes more reliable in adhering to a blood glucose management regime. Once the first award has been provided to the first user, e.g., once an electronic message including a link to a gift card has been transmitted, or once a check or monetary award has been deposited in a bank account associated with the user, the monitoring system may record that the first award has been received.

Where the user has not satisfied all of the requirements for earning a first award, the systems and methods of the present disclosure may determine whether the user requires medical intervention, or whether the motivation strategy is appropriate for the user. At box 552, whether the blood glucose reading satisfies an intervention threshold may be determined. For example, the blood glucose reading may indicate a level of glucose in the blood that is not only outside of a preferred range but is also dangerous (e.g., above 400 mg/dl or below 60 mg/dl), or that the blood glucose reading was obtained at an unacceptably late point in time, e.g., more than twenty-four hours later than required. If the blood glucose reading satisfies the intervention threshold, then the process proceeds to box 564, where a message indicating that the user requires assistance is transmitted, e.g., to the user, or to an interested party or third party, and to box 590, where information regarding the blood glucose reading is stored in at least one data store. The information may include not only a blood glucose level or related information but also the threshold that was exceeded or the content of the message transmitted to the user or to the interested party or third party.

If the blood glucose reading did not satisfy an intervention threshold, the process proceeds to box 562, where a monitoring system may determine whether another award is more appropriate for the user in accordance with the motivation strategy. For example, one reason why the user failed to satisfy the requirements for earning the first award may be that the first award does not provide enough of an incentive to the user. If the user is a child who collects hockey cards and baseball cards, a motivation strategy may incorporate one or more incentives relating to his or her interest in such cards. If the first award includes one or more hockey cards, the user may be initially motivated to comply with the blood glucose management regime for the purpose of earning hockey cards, but may become less motivated after earning a sufficient number of hockey cards, to the point where the motivation to earn more hockey cards is diminished. Therefore, if another award may be more appropriate for providing a sufficient incentive to the user, then the process may advance to box 574, where a second award is designated for the user in accordance with the motivation strategy, and to box 590, where the information regarding the blood glucose reading is stored in at least one data store. For example, if it is determined that hockey cards are no longer a viable award in accordance with the motivation strategy, baseball cards may be substituted for hockey cards as a second award for complying with the blood glucose management regime.

After a user has received the first award, or if the user fails to satisfy all requirements for earning the first award but where no other award is identified as appropriate for the user in accordance with the motivation strategy, then the process advances to box 580, where the monitoring system determines whether the motivation strategy is sufficient to accomplish the goals of the blood glucose management regime. For example, if the motivation strategy is intended to provide incentives for complying with a blood glucose management regime based on blood glucose levels identified in daily readings, and the user either regularly satisfies the requirements for earning the first award based on his or her daily readings, or is unable to earn or uninterested in earning the first award based on such readings, then the strategy may be focused on ensuring that blood glucose levels in readings taken at more frequent (e.g., hourly) or less frequent (e.g., weekly) intervals satisfy requirements for earning the first award. Similarly, if the motivation strategy is directed at minimizing a number of non-compliant blood glucose readings, and the user either regularly minimizes the number, or is unable to meet a threshold associated with the number, then a motivation strategy that focuses on specific blood glucose levels may be defined.

If the current motivation strategy is deemed sufficient, then the process advances to box 590, where information regarding the blood glucose reading is stored in at least one data store. If the current motivation strategy is not deemed sufficient, then the process advances to box 582, where an alternate motivation strategy may be selected for the user based on his or her attributes, and to box 590, where information regarding the blood glucose reading is stored in at least one data store.

Accordingly, the systems and methods of the present disclosure may utilize one or more strategies for motivating diabetics to comply with a blood glucose management regime, and may offer one or more awards or incentives to the diabetics when the diabetics meet all relevant requirements of a motivation strategy. Where an award or an incentive is no longer appropriate or attractive to a user, or where the motivation strategy is no longer effective, the systems and methods disclosed herein may substitute new awards or incentives, or identify alternative motivation strategies, which may increase not only the effectiveness of a blood glucose management regime but also a diabetic's willingness to adhere to its requirements.

As mentioned above, the system and methods of the present disclosure may make one or more predictions of a blood glucose level of a diabetic based on one or more attributes of the diabetic based on the diabetic's past experiences and history when having such attributes. For example, where a diabetic is determined to be in a given location, or performing a given activity, the systems and methods disclosed herein may predict a blood glucose level of the diabetic based on his or her historical blood glucose levels while in the location or when participating in the activity. Once a prediction has been made as to a blood glucose level of the diabetic based on such attributes, the systems and methods disclosed herein may further provide one or more messages to a diabetic, to interested parties associated with the diabetic, or to other third parties including information regarding a prediction of the diabetic's blood glucose level. Such messages may indicate whether the prediction is favorable or unfavorable, and may include any number or type of information or data regarding the prediction, including instructions for addressing the diabetic's blood glucose level in the event that the prediction is deemed accurate.

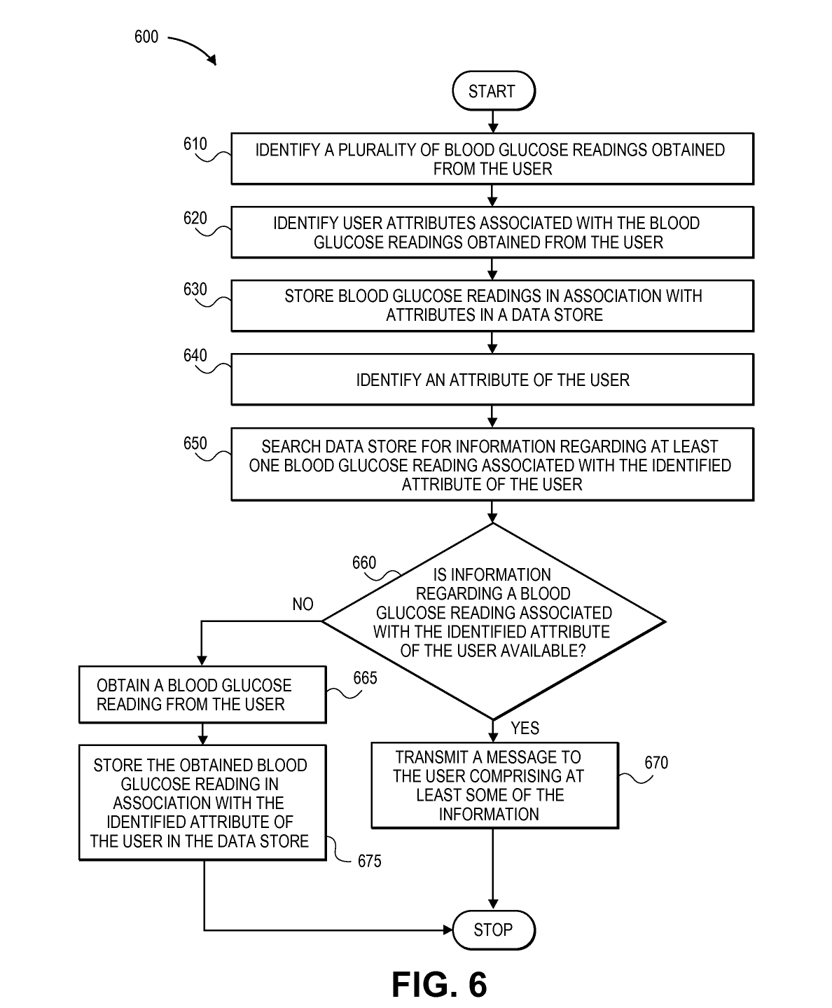

Referring to FIG. 6, a flow chart 600 of one process for monitoring blood glucose levels is shown. At box 610, a plurality of blood glucose readings obtained from the user are identified. The readings may have been obtained at various times using a blood glucose meter, such as the blood glucose meter 120 of FIG. 1, and transmitted to the monitoring system 170 over the network 190. At box 620, user attributes associated with the blood glucose readings are identified. The attributes may relate to the user's diabetic condition, as well as his or her height or weight, times of day or locations, any medications that were prescribed to the user, any medical devices or equipment that were available to the user, any activities in which the user was participating, or any other information or data relating to the user at the times when such readings were obtained. At box 630, the blood glucose readings are stored in association with the user attributes in a data store. For example, the readings and the attributes may be stored in an index, a record or other set of information or data maintained in the data store.

At box 640, an attribute of the user is identified. The attribute may be a current height or weight of the user, as well as the current time of day or a current location of the user, any medications currently prescribed to the user, any medical devices or equipment currently available to the user, or any activities in which the user is currently participating, or any other information or data currently relating to the user. At box 650, the data store is searched for information regarding at least one blood glucose reading associated with the user attribute identified at box 640. The monitoring system may query the data store into which the blood glucose readings were stored in association with the user attributes at box 630, in an effort to identify any blood glucose readings that may be associated with the user attribute identified at box 640. For example, the monitoring system may search for blood glucose readings that were obtained at the same or at similar times of day, or while the user was in the same or in similar locations, or participating in similar activities.

At box 660, whether information regarding received a blood glucose reading associated with the identified attribute is available is determined. If no such blood glucose readings associated with the attribute identified at box 640 are available, then the process advances to box 665, where a blood glucose reading is obtained from the user, and to box 675, where the blood glucose reading is stored in association with the identified attribute of the user in the data store, and the process ends. For example, if the user does not have any available information regarding blood glucose readings taken at a certain time of day, in a given location, or during or following the performance of a specific activity, then the user may be prompted to obtain a blood glucose reading, and information regarding the blood glucose reading (e.g., a measured blood glucose level) may be stored in association with the time of day, the given location or the specific activity in the data store.

If a blood glucose reading associated with the attribute of the user identified at box 640 is available, then the process advances to box 670, where a message comprising at least some of the information regarding the blood glucose reading is transmitted to the user, and the process ends. For example, if the user was previously observed to have an unacceptably low blood glucose level at a certain time of day, then a message may be transmitted to the user, or to an interested party or third party, reminding the user to consume food, as necessary, at that time of day. If the user was previously observed to have a high blood glucose level when visiting a specific location, e.g., a restaurant or recreational facility, a message may be transmitted to the user upon determining that the user is at the specific locations. Such messages may include any relevant information regarding the user or a relevant attribute, as well as the user's observed blood glucose levels as identified in blood glucose readings that were previously taken and associated with the relevant attribute.

Accordingly, the systems and methods of the present disclosure may make one or more predictions as to a diabetic's blood glucose level based on an observed attribute associated with the user, e.g., a height or weight of the user, a time of day, or any activities in which the user is participating, which may be compared to an index, record or other set of information regarding blood glucose readings that were previously obtained from the user and stored in association with one or more user attributes in one or more data stores. Where the observed attribute is consistent with one or more of the user attributes, the previously obtained blood glucose reading associated with the observed attribute may be identified, and information regarding the previously obtained blood glucose reading may be provided to the user in one or more messages.

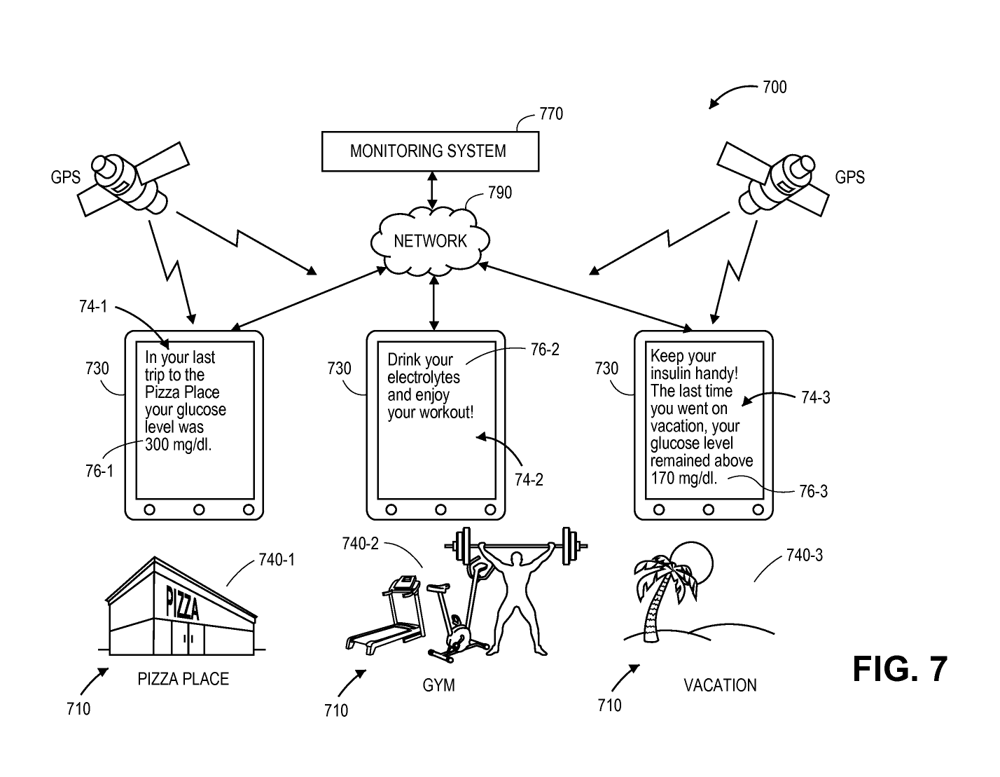

In particular, the systems and methods of the present disclosure may make predictions as to a diabetic's blood glucose level based on his or her location, or during the performance of a specific activity, which may be compared to blood glucose readings previously obtained from the diabetic at that location, or in similar locations, or during the performance of the same activity, or similar activities. Referring to FIG. 7, a block diagram of a computerized system 700 for monitoring blood glucose levels is shown. The system 700 includes a user 710 of a communications device 730 (e.g., a smartphone), a monitoring system 770 and a network 790. The communications device 730 is configured to determine a location from one or more Global Positioning System satellites or by any other means, and the location may be associated with the user 710. As is shown in FIG. 7, the user 710 may transport the communications device 730 to a variety of locations, such as a pizza place 740-1, a gymnasium 740-2 and a vacation destination 740-3.

The monitoring system 770 of FIG. 7 is configured to receive information regarding an attribute of the user 710, e.g., a location of the user 710, or an activity being performed by the user 710, and to send a message 74 to the communications device 730 concerning a previously measured blood glucose level 76 of the user 710 that is associated with the identified attribute, e.g., the location of the user 710, or the activity being performed by the user 710.

For example, as is shown in FIG. 7, when the monitoring system 770 determines that the user 710 is at the pizza place 740-1, the monitoring system 770 identifies information regarding a blood glucose reading previously obtained from the user 710 following a visit to the pizza place 740-1, and transmits a message 74-1 including information 76-1 regarding a blood glucose level of the user 710 following a previous visit to the pizza place 740-1 to the communications device 730. When the monitoring system 770 determines that the user 710 is exercising at the gymnasium 740-2, the monitoring system 770 identifies information regarding a blood glucose reading previously obtained from the user 710 after exercising at the gymnasium 740-2, and transmits a message 74-2 including a reminder 76-2 to consume electrolytes during his or her workout to the communications device 730. When the monitoring system 770 determines that the user is on vacation 740-3, the monitoring system 770 identifies information regarding one or more blood glucose readings previously obtained from the user 710 during a previous vacation, and transmits a message 74-3 including information 76-3 regarding blood glucose levels of the user 710 during the previous vacation to the communications device 730.

Accordingly, the systems and methods of the present disclosure may identify an attribute of a user, and predict a blood glucose level of the user by comparing the attribute of the user to previously observed attributes of the user and identifying a blood glucose reading associated with one or more of the previously observed attributes. Where the identified attribute is consistent with one or more of the previously observed attributes, a prediction of the blood glucose level of the user may be determined based on a blood glucose reading associated with the one or more previously observed attributes. Information regarding the predicted blood glucose level may be provided to the user in one or more messages, such as the messages 74-1, 74-2, 74-3 of FIG. 7, or to one or more interested parties or third parties associated with the user. A prediction as to a user's blood glucose level may be derived based on any number or type of attributes of the user, including but not limited to a location of the user, or an activity in which the user is participating.

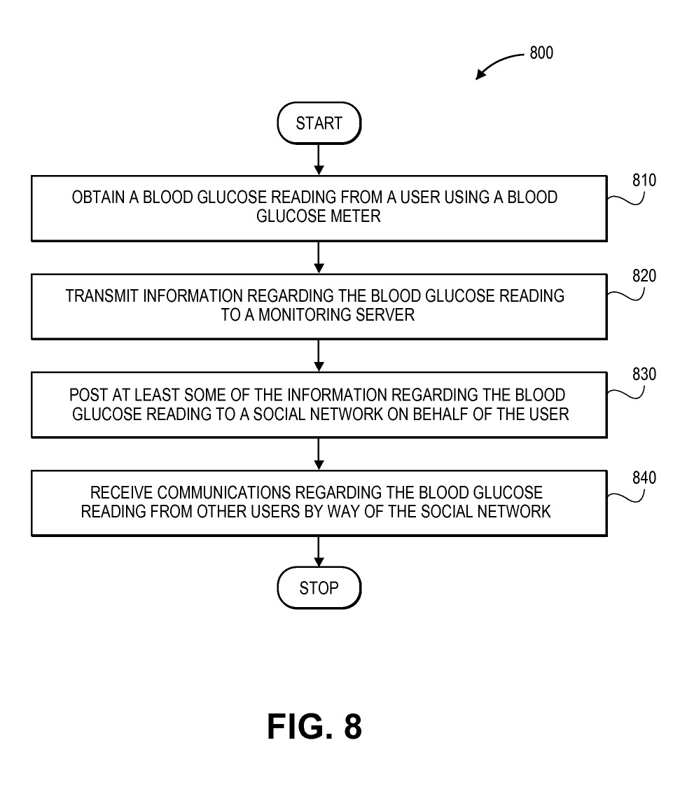

Additionally, as is also mentioned above, the system and methods of the present disclosure may further enhance the ability of a diabetic to adhere to a blood glucose management regime by enabling the diabetic to draw motivational support from interested parties associated with the diabetic or other third parties, e.g., other diabetics, by way of a social network. Referring to FIG. 8, a flow chart 800 of one process for monitoring blood glucose levels is shown. At box 810, a blood glucose meter is used to obtain a blood glucose reading from a user. For example, referring to the system 100 of FIG. 1, a blood glucose reading may be obtained from the user 110 using the blood glucose meter 120. At box 820, information regarding the blood glucose reading is transmitted to a monitoring system. For example, referring again to the system 100 of FIG. 1, any relevant information regarding the blood glucose reading (e.g., a measured blood glucose level, a timestamp, a geotag or any other user-related information) is transmitted over the network 190 to the monitoring system 170, where the information may be stored in any type or form of data store and subjected to any type or form of analysis.

At box 830, at least some of the information regarding the blood glucose reading is posted to a social network on behalf of the user. For example, according to some embodiments of the present disclosure, the information may be manually posted to the social network by the user, or by an interested party or third party associated with the user (e.g., family members, physicians or other medical personnel or other diabetics). Alternatively, the information may be automatically posted to the social network by the monitoring system, which may be provided with any number of authorizations or permissions required to directly access the social network on behalf of the user, e.g., through one or more plug-ins or like software components or modules. The information may include the blood glucose level included in the blood glucose reading, as well as any other relevant information or data regarding the user or the blood glucose reading.

At box 840, the user receives communications in response to the posted information regarding the blood glucose reading from other users by way of the social network. Such other users may be designated as social network connections of the user (e.g., “friends” or “followers”), or of the monitoring system. Alternatively, the other users may be identified as sharing one or more user attributes in common with the user. For example, the user and the other users may have the same or similar ages or genders (e.g., forty-year-old diabetic females), or may be located in the same general vicinity (e.g., Type 1 diabetics located in the state of Alaska). The communications provided by such other users may be made privately or publicly, e.g., in the form of public postings or private messages, and may include any relevant information intended for the user, such as words of encouragement, comfort or countenance.

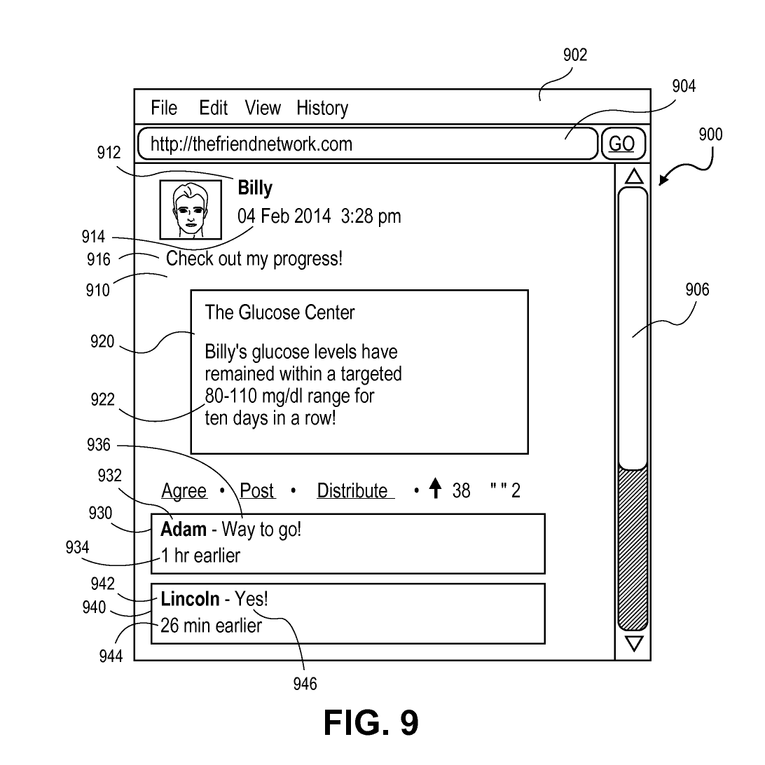

Therefore, the systems and methods of the present disclosure may further enable a diabetic to receive motivation through one or more social networks, which may receive information regarding a blood glucose reading of a user, and post at least some of the information to such networks on behalf of the diabetic, or through an account maintained by the diabetic. One such social network posting is shown in FIG. 9. Referring to FIG. 9, a user interface 900 (e.g., a browser) displayed by one or more systems or methods for monitoring blood glucose levels is shown. The user interface 900 includes a menu bar 902, an address bar 904 and a slider 906 for providing or translating content within the user interface 900. Additionally, the user interface 900 further includes information regarding a social network posting 910 made on behalf of a user 912.

The social network posting 910 may include a name of the user 912, as well as a time stamp 914 associated with the social network posting 910. Alternatively, the social network posting 910 may further include information regarding a location of the user 912 at a time when the social network posting 910 was made. Additionally, the social network posting 910 may further include a set of text 916 or other information provided by the user 912. The set of text 916 provided by the user 912 may further relate to a blood glucose reading or include any other relevant information or data regarding the user. Those of ordinary skill in the pertinent art will recognize that the social network posting 910 may have been made by the user 912 directly, e.g., through the use of a smartphone, a tablet computer, a laptop computer or a desktop computer, or any other like device, or on behalf of the user 912 by a monitoring system or other external source (not shown), with the authorization or permission of the user 912.

The social network posting 910 may also include information 920 regarding a blood glucose reading obtained from the user 912, which may be determined or provided by a monitoring system or other external source. The information 920 may further include information 922 regarding a level of blood glucose in the blood of the user 912, or any other relevant information or data regarding the blood glucose reading provided by the monitoring system (e.g., a timestamp, a geotag or any other intrinsic or extrinsic information or data relating to the user).

Moreover, those of ordinary skill in the pertinent arts will also recognize that other users of the social network may provide one or more messages or postings in response to the social network posting 910 provided by the user 912. As is shown in FIG. 9, the social network posting 910 includes postings 930, 940 by other users 932, 942 of the social network. The postings 930, 940 may include, for example, words of encouragement to the user 912 based on his or her participation in a blood glucose management regime, as well as words of praise for achieving one or more goals or objectives, or receiving one or more awards or incentives, for his or her compliance with the blood glucose management regime, or any other relevant information or data.

For example, a diabetic user of a social network (e.g., a teenager) may identify or designate one or more friends, colleagues or fellow diabetics as his or her connections on the social network. When the diabetic user obtains a blood glucose reading using a blood glucose meter or other glucometer, information regarding the blood glucose reading may be transmitted to a monitoring system for recording and analysis. After the information regarding the blood glucose reading has been recorded and analyzed, e.g., for compliance with one or more requirements of a blood glucose management regime, or for any other purpose, the monitoring system may transmit a message to the diabetic user indicating whether the blood glucose reading complies with the blood glucose management regime. Such information may be transmitted through any form of message, including but not limited to an E-mail message, a text message or a message over the social network.

Upon receiving information regarding his or her blood glucose readings, the diabetic user may elect to make a posting to the social network, such as the social network posting 910 of FIG. 9, indicating his satisfaction or displeasure with his or her blood glucose readings, and one or more of the diabetic user's social network connections, such as the users 932, 942, may reply in kind with postings of their own (e.g., the postings 930, 940). After receiving words of encouragement or praise from the users 932, 942, the user 912 may feel more motivated to continue to adhere to the blood glucose management regime. Likewise, where one or more of the users 932, 942 is also a diabetic, such users 932, 942 may also feel motivated to adhere to their own blood glucose management regimes.

Accordingly, the systems and methods of the present disclosure may utilize the power and prevalence of one or more social network outlets to provide information regarding a user's blood glucose readings to the user, or to connections of the user, which may include interested parties such as parents or family members, as well as third parties such as doctors, medical staff or connections of the user on the social network. The systems and methods of the present disclosure may further permit the user to receive words of encouragement and support from his or her connections by way of the social network, thereby enhancing his or her level of motivation to comply with a blood glucose management regime, and to maintain the glucose in his or her blood at acceptable levels.

Although the disclosure has been described herein using exemplary techniques, components, and/or processes for implementing the systems and methods of the present disclosure, it should be understood by those skilled in the art that other techniques, components, and/or processes or other combinations and sequences of the techniques, components, and/or processes described herein may be used or performed that achieve the same function(s) and/or result(s) described herein and which are included within the scope of the present disclosure.

For example, although some of the embodiments disclosed herein reference the taking of blood samples or blood glucose readings at a blood glucose meter, and the analysis of information regarding such samples or readings at a discrete, external monitoring system, the systems and methods disclosed herein are not so limited. Rather, blood samples or blood glucose readings may be taken from a diabetic at a blood glucose meter, and analyzed with regard to one or more requirements of a blood glucose management regime at the blood glucose meter, or at an associated communications device (or relay device), and any information regarding the compliance of such samples or readings with the requirements of the blood glucose management regime may be transmitted directly from the blood glucose meter or communications device (or relay device) to a network, and to one or more computer devices associated with the diabetic, or with one or more interested parties or third parties associated with the diabetic, in accordance with the present disclosure.

Moreover, although some of the embodiments described herein include specific systems or methods for obtaining blood glucose readings or communicating information regarding such readings to one or more individuals or entities, the systems and methods of the present disclosure are not so limited, and may be used with any means or method for obtaining blood glucose readings or computer-based communication. For example, although some embodiments of the present disclosure reference obtaining a blood glucose reading by pricking a portion of a human body and analyzing blood extracted from the site of the prick, any type or from of blood glucose meter or blood glucose analysis device may be used in accordance with the present disclosure, including blood glucose meters that analyze samples of blood as well as bloodless glucose meters or glucometers. Additionally, although some other embodiments of the present disclosure describe predicting a blood glucose level of a user based on his or her location, the systems and methods disclosed herein are not so limited, and may be used to predict a blood glucose level of the user based on a location of the user as determined by any other means (e.g., network signal triangulation), or on any other attribute. Such means or methods may be used in series or in parallel, and independently or in conjunction with one another, in accordance with the present disclosure.

It should be understood that, unless otherwise explicitly or implicitly indicated herein, any of the features, characteristics, alternatives or modifications described regarding a particular embodiment herein may also be applied, used, or incorporated with any other embodiment described herein, and that the drawings and detailed description of the present disclosure are intended to cover all modifications, equivalents and alternatives to the various embodiments as defined by the appended claims. Moreover, with respect to the one or more methods or processes of the present disclosure described herein, including but not limited to the flow charts shown in FIG. 6 or 8, the order in which the steps of the methods or processes are listed is not intended to be construed as a limitation on the claimed inventions, and any number of the method or process steps can be combined in any order and/or in parallel to implement the methods or processes described herein. Also, the drawings herein are not drawn to scale.

Conditional language, such as, among others, “can,” “could,” “might,” or “may,” unless specifically stated otherwise, or otherwise understood within the context as used, is generally intended to convey in a permissive manner that certain embodiments could include, or have the potential to include, but do not mandate or require, certain features, elements and/or steps. In a similar manner, terms such as “include,” “including” and “includes are generally intended to mean “including, but not limited to.” Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” or “at least one of X, Y and Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Although the invention has been described and illustrated with respect to exemplary embodiments thereof, the foregoing and various other additions and omissions may be made therein and thereto without departing from the spirit and scope of the present disclosure.

What is claimed is:

1. A mobile computer system for monitoring blood glucose levels comprising:
a glucose meter configured to obtain at least one blood glucose reading from a user; and
a communications device configured to automatically transmit information associated with the at least one blood glucose reading to a monitoring system.

2. The mobile computer system of claim 1, further comprising a relay device configured to:
automatically receive the information associated with the at least one blood glucose reading from the communications device; and
automatically transmit at least some of the information associated with the at least one blood glucose reading to the monitoring system.

3. The mobile computer system of claim 2, wherein the relay device is further configured to display a log comprising at least some of the information received from the communications device on at least one computer display.

4. The mobile computer system of claim 1, wherein the information associated with the at least one blood glucose reading comprises at least one of:
a measured blood glucose level of the user;
a timestamp;
a geotag; or
user-generated information associated with the blood glucose reading.

5. A computer-implemented method for monitoring blood glucose levels comprising:
receiving information associated with a first blood glucose reading obtained from a user;
analyzing at least some of the information associated with the first blood glucose reading obtained from the user;
generating a first electronic message based at least in part on the analyzed information; and
providing the first electronic message to at least one of:
the user; or
a party associated with the user.

6. The computer-implemented method of claim 5, wherein analyzing the at least some of the information associated with the first blood glucose reading obtained from the user comprises:
identifying, at a monitoring system, a blood glucose management regime comprising at least one requirement; and
comparing, at the monitoring system, at least some of the analyzed information to the at least one requirement.

7. The computer-implemented method of claim 6, wherein analyzing the at least some of the information associated with the first blood glucose reading obtained from the user further comprises:
identifying, at the monitoring system, at least one element of the analyzed information that does not satisfy the at least one requirement.

8. The computer-implemented method of claim 7,
wherein the at least one requirement is a compliant blood glucose range;
wherein the at least one element is a measured blood glucose level of the user that is substantially outside the compliant blood glucose range; and
wherein the first electronic message comprises an indication that the measured blood glucose level of the user is substantially outside the compliant blood glucose range.

9. The computer-implemented method of claim 7,
wherein the at least one requirement is a predetermined interval of time;
wherein the at least one element is a timestamp indicating that the information associated with the first blood glucose reading obtained from the user was received by the monitoring system substantially outside the predetermined interval of time; and
wherein the first electronic message comprises at least one of:
an indication that the information associated with the first blood glucose reading obtained from the user was received by the monitoring system substantially outside the predetermined interval of time; or
an indication that a second blood glucose reading is required;

10. The computer-implemented method of claim 7, wherein identifying the at least one element of the analyzed information that does not satisfy the at least one requirement further comprises:
detecting, at the monitoring system, whether the at least one element exceeds a predetermined intervention threshold,
wherein, if the at least one element exceeds the predetermined intervention threshold, the first electronic message comprises an indication that the user requires assistance.

11. The computer-implemented method of claim 6, wherein analyzing the at least some of the information associated with the first blood glucose reading obtained from the user further comprises:
identifying at least one element of the analyzed information that satisfies the at least one requirement.

12. The computer-implemented method of claim 11, further comprising:
identifying a first attribute of an at least one attribute of the user;
selecting, based at least in part on the first attribute, a first motivation strategy comprising a first plurality of awards; and
selecting a first award of the first motivation strategy based at least in part on the at least one element;
wherein the first electronic message comprises an indication that user qualifies for the first award.

13. The computer-implemented method of claim 12, further comprising:
selecting, based at least in part on the first attribute, a second motivation strategy comprising a second plurality of awards;
selecting a second award of the second motivation strategy based at least in part on the at least one element;
generating a second electronic message comprising an indication that the user qualifies for the second award; and
providing the second electronic message to at least one of:
the user; or
the party associated with the user.

14. The computer-implemented method of claim 12, further comprising:
identifying a second attribute of the at least one attribute of the user;
selecting, based at least in part on the second attribute, a second motivation strategy comprising a second plurality of awards;
selecting a second award of the second motivation strategy based at least in part on the at least one element;
generating a second electronic message comprising an indication that the user qualifies for the second award; and
providing the second electronic message to at least one of the following:
the user; or
the party associated with the user.

15. The computer-implemented method of claim 12, wherein the at least one attribute of the user comprises at least one of:
an age of the user;
a gender of the user;
a race of the user;
a height of the user;
a weight of the user;
a location of the user;
a medical device used by the user;
a medication taken by the user;
a hobby of the user;
a family history of the user; or
genetic information regarding the user.

16. The computer-implemented method of claim 5, wherein the information associated with the first blood glucose reading obtained from the user comprises at least one of:
a measured blood glucose level of the user;
a timestamp;
a geotag; or
user-generated information associated with the first blood glucose reading.

17. The computer-implemented method of claim 5,
wherein the information associated with the first blood glucose reading obtained from the user includes a first value of an attribute of the user at a first time,
wherein the first blood glucose reading was obtained from the user at the first time; and
wherein analyzing the at least some of the information associated with the first blood glucose reading obtained from the user further comprises:
identifying a second value of the attribute of the user at a second time,
determining whether the second value of the attribute is consistent with the first value of the attribute; and
upon determining that the second value of the attribute is consistent with the first value of the attribute,
generating the first electronic message based at least in part on one of the first value of the attribute of the user or the first blood glucose reading.

18. The computer-implemented method of claim 17, wherein the first value of the attribute is based at least in part on a measured blood glucose level of the user.

19. A computer-implemented method for monitoring blood glucose levels comprising:
obtaining, by way of a blood glucose meter, at least one blood glucose reading from a first user having a user attribute;
automatically transmitting, by way of a mobile computing device associated with the blood glucose meter, information associated with the at least one blood glucose reading to a monitoring system;
making a posting, by way of the mobile computing device, to a first social network account associated with the first user,
wherein the posting comprises at least some of the information associated with the at least one blood glucose reading;

20. The computer-implemented method of claim 19, further comprising:
identifying, by way of the first social network account associated with the first user, a second social network account associated with a second user, based at least in part on one of:
the at least some of the information associated with the at least one blood glucose reading; or
the user attribute of the first user.

21. The computer-implemented method of claim 20, further comprising:
receiving, at the mobile computing device, a message from the second user,
wherein the message is based at least in part on the posting.

22. The computer-implemented method of claim 19, wherein the user attribute of the first user comprises at least one of:
a diabetic condition;
an age of the first user;
a gender of the first user;
a race of the first user;
a height of the first user;
a weight of the first user;
a location of the first user;
a medical device used by the first user;
a medication taken by the first user;
a hobby of the first user;
a family history of the first user; or
genetic information regarding the first user.
